(19) United States
(12) Patent Application Publication
(10) Pub. No.: US 2008/0039769 A1
(43) Pub. Date: Feb. 14, 2008

(54) METHOD OF MEDICAL TREATMENT USING CONTROLLED HEAT DELIVERY

(75) Inventor: Gholam A. Peyman, Sun City, AZ (US)

Correspondence Address: BELL, BOYD, & LLOYD LLP, P.O. BOX 1135, CHICAGO, IL, 60690, US

(73) Assignee: MINU LLC, Pittsboro, NC (US)

(21) Appl. No.: 11/562,268

(22) Filed: Nov. 21, 2006

Related U.S. Application Data
(63) Continuation-in-part of application No. 09/986,141, filed on Nov. 7, 2001, now Pat. No. 6,918,904.
Application No. 09/986,141 is a continuation-in-part of application No. 11/070,659, filed on Mar. 2, 2005.
Application No. 11/070,659 is a continuation-in-part of application No. 11/446,065, filed on Jun. 1, 2006.
Application No. 11/446,065 is a continuation-in-part of application No. 11/558,788, filed on Nov. 10, 2006.

(51) Int. Cl. A61F 9/00; A61F 7/00; A61M 37/00

(52) U.S. Cl. 604/20; 604/291; 606/27; 607/113

(57) ABSTRACT

The present invention relates to a system for treating a patient. The system includes a device configured to be positioned adjacent a portion of the patient, a medicinal substance delivery device, adapted to delivery a medicinal substance to the portion of the patient, a heating means adapted to provide heat to the device and a control system configured to monitor the heating means and control the temperature of the device at the portion of the patient.

### RELATED APPLICATIONS

This application is a continuation-in-part of U.S. patent application Ser. No. 09/986,141, filed Nov. 7, 2001, entitled “Method of Reshaping the Cornea by Controlled Thermal Delivery”, and U.S. patent application Ser. No. 11/070,659 filed Mar. 2, 2005, entitled “Device and Method for Reshaping the Cornea”, the entire contents of both of which are incorporated herein by reference.

This application is related to U.S. patent application Ser. No. 11/446,065, filed Jun. 1, 2006 and entitled “Device and Method for Reshaping the Cornea”, and U.S. patent application Ser. No. 11/558,788, filed Nov. 10, 2006 and entitled “Method of Treating the Eye using Controlled Heat Delivery,” the entire contents of both of which are incorporated herein by reference.

### DESCRIPTION OF RELATED PRIOR ART

The most common type of glaucoma is primary open angle glaucoma (POAG). One factor in the cause of glaucoma is obstruction of the outflow of aqueous humour from the eye. Aqueous humour is produced by the epithelium lining the eye's ciliary body which then flows through the pupil and into the anterior chamber. The trabecular meshwork then drains the humour to Schlem's canal, and ultimately to the venous system. All eyes have some intraocular pressure, which is caused by some resistance to the flow of aqueous through the trabeculum and Schlem's canal. Pressures of anywhere between 7 and 21 mm Hg are considered normal. If the intraocular pressure is too high, (>21.5 mm Hg), the pressure exerted on the walls of the eye results in compression of the ocular structures. The portions of the trabecular meshwork can become blocked or plugged, thus causing an increase in intraocular pressure.

To treat these blockages, Glaucoma drainage devices, also known as tube shunts, are implanted that are designed to maintain an artificial drainage pathway. A small incision is made in the conjunctiva, usually towards the top of the eye. The surgeon will then make a tiny incision in the sclera of the eye and will fashion an opening for the drainage implant device. The drainage tube will be placed such that the opening of the tiny tube is inside the anterior chamber of the eye where it is bathed in aqueous fluid. The tube is sutured in place with the drainage device attached to the sclera of the eye. Most surgeons will place an absorbable suture around the tube at the time of surgery to prevent filtration through the device until a fibrous capsule has formed. As such, the device is not expected to function until about 3 to 8 weeks following the procedure.

Other types of ailments of the eye are choroidal tumors, melanoma and retinoblastoma. To treat these ailments, radiation can be used. Radiation, at the appropriate dose rates and in the proper physical forms, is intended to eliminate growing tumor cells without causing damage to normal tissue sufficient to require removal of the eye. As the cells die, the tumor shrinks.

Another way to treat tumors is using high energy particles (helium ion or proton beam radiation) from a cyclotron to irradiate the tumors. Surgery is performed first to sew small metal clips to the sclera so that the particle beam can be aimed accurately. Treatment is given over several successive days.

Other treatments have been used for a small number of patients. Photocoagulation using white light or laser light has been used to bum small tumors, and cryo-therapy has been used to kill the tumors by freezing them. A few patients have had eye wall resection or a related procedure to remove tumors from their eyes.

Age-related macular degeneration (ARMD) is the leading cause of blindness among persons over fifty in the United States and other countries. Two forms of age-related macular degeneration are known: (1) neovascular, also known as exudative, age-related macular degeneration (E-ARMD) and (2) nonneovascular, also known as nonexudative, age-related macular degeneration (NE-ARMD). NE-ARMD is characterized by the presence of drusen, yellow-white lesions of the retinal pigment epithelium within the macula, and by other abnormalities of the retinal pigment epithelium, including retinal cell death.

Although the exact etiology of ARMD is not known, several risk factors seem to be important for the manifestation of this disease. For example, ARMD may be caused by chronic exposure of the retina to light. The presence or absence of certain nutrients in the diet, such as the antioxidant vitamins E and C, also may affect one's predisposition for ARMD. Other conditions, such as hypertension and smoking, are also considered to be important risk factors for the development of this disease.

Several therapeutic methods have been tried. For example, vitamins and dietary supplements have been used for the purpose of delaying the onset of disease. Thalidomide is being investigated to determine if it will slow down or arrest new vessel formation. Laser or radiation has been used to destroy new vessels. However, none of these methods has led to successful results and no definitive treatment for ARMD has been developed to date.

Other methods have been used to delivery drugs to portions of the human body. For example, several iontophoresis drug delivery systems have used. These devices can include a device which is attachable to the skin of an animal and has a drug reservoir, an electrolyte reservoir, and electrodes electrically communicating with the reservoirs and with the skin of the animal. An electronic circuit for controlling the rate or amount of current or voltage applied to the electrodes of the device includes a constant voltage and current generator, and a switching circuit for switching between applying either a constant current to the electrodes or a constant voltage. Current and voltage sensors are attached to the electrodes and provide feedback signals which are compared with predetermined threshold signals to control through feedback the amount of constant voltage and constant current applied to the electrodes.

### SUMMARY OF THE INVENTION

The present invention relates to a system for treating a patient. The system includes a device configured to be positioned adjacent a portion of the patient, a medicinal substance delivery device, adapted to delivery a medicinal substance to the portion of the patient, a heating means adapted to provide heat to the device and a control system configured to monitor the heating means and control the temperature of the device at the portion of the patient.

The present invention also relates to a method of treating a patient, including the steps of positioning a device adjacent a portion of a patient, delivering a medicinal substance to the portion of the patient, heating the device, monitoring the temperature at the portion of the patient, and controlling the temperature of the device.

The present invention also relates to a system for treating a patient, including a device configured to be implanted in a portion of an animal, a heating means for heating the device to an appropriate temperature, and a control system configured to monitor the heating means and control the temperature of the device at the portion of the patient.

The present invention also relates to a method for treating a patient, including the steps of implanting a device in a portion of an animal, heating the device to an appropriate temperature, monitoring the temperature of the device, and controlling the temperature of the device at the portion of the patient.

Other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 is a side elevational view in cross section taken through the center of an eye showing the cornea, pupil and lens;

FIG. 2 is a side elevational view in cross section of the eye of FIG. 1 with a flap formed in the surface of the cornea;

FIG. 3 is a side elevational view in cross section of the eye of FIG. 2 with a reshaping device having a predetermined shape for correcting myopia proximate to the exposed surface of the cornea;

FIG. 4 is a side elevational view in cross section of the eye of FIG. 3 with the reshaping device immediately adjacent and overlying the exposed surface of the cornea;

FIG. 5 is a side elevational view in cross section of the eye of FIG. 4 with a laser irradiating the reshaping device to soften the cornea with the softened portion of the cornea conforming to the internal shape of the reshaping device;

FIG. 6 is a side elevational view in cross section of the eye of FIG. 5 with the reshaping device removed and the cornea maintaining its reformed shape;

FIG. 7 is a side elevational view in cross section of the eye of FIG. 6 with the flap repositioned over the reformed exposed surface of the cornea;

FIG. 8 is a side elevational view in cross section of the eye of FIG. 2 with a reshaping device having a predetermined shape for correcting hyperopia proximate to the exposed surface of the cornea;

FIG. 9 is a side elevational view in cross section of the eye of FIG. 8 with the reshaping device immediately adjacent and overlying the exposed surface of the cornea;

FIG. 10 is a side elevational view in cross section of the eye of FIG. 9 with a laser irradiating the surface of the cornea to soften the cornea with the softened portion of the cornea conforming to the internal shape of the reshaping device;

FIG. 11 is a side elevational view in cross section of the eye of FIG. 10 with the reshaping device removed and the cornea maintaining its reformed shape;

FIG. 12 is a side elevational view in cross section of the eye of FIG. 11 with the flap repositioned over the reformed exposed surface of the cornea;

FIG. 13 is a side elevational view in cross section of the eye of FIG. 2 with a thermally conductive reshaping device having a predetermined shape immediately adjacent the exposed surface of the cornea;

FIG. 14 is a side elevational view in cross section of the eye of FIG. 13 with the thermally conductive reshaping device administering controlled heat to the exposed surface of the cornea to soften the cornea with the softened portion of the cornea conforming to the internal shape of the reshaping device;

FIG. 15 is a side elevational view in cross section of the eye of FIG. 2 with a reshaping device having two passageways for irrigation and aspiration of a liquid with a predetermined temperature and having a predetermined shape immediately adjacent the exposed surface of the cornea;

FIG. 16 is a side elevational view in cross section of the eye of FIG. 15 with the aspiration and irrigation tubes extending through the reshaping device for administering and removing liquid with a predetermined temperature to the exposed surface of the cornea to soften the cornea with the softened portion of the cornea conforming to the internal shape of the reshaping device;

FIG. 17 is a side elevational view in cross section of the eye of FIG. 2 with a inlay positioned on the exposed surface of the cornea and with a reshaping device having a predetermined shape for correcting myopia proximate to the inlay;

FIG. 18 is a side elevational view in cross section of the eye of FIG. 17 with the reshaping device immediately adjacent the inlay;

FIG. 19 is a side elevational view in cross section of the eye of FIG. 18 with a laser irradiating the lens to soften the inlay with the softened portion of the inlay conforming to the internal shape of the lens;

FIG. 20 is a side elevational view in cross section of the eye of FIG. 19 with the lens removed and the flap repositioned over the reformed inlay;

FIG. 21 is a side elevational view in cross section of the eye of FIG. 1 with multiple cavities formed in the cornea via an ultra short pulse laser;

FIG. 22 is a front view of the eye of FIG. 21 showing the multiple cavities forming a substantially circular pattern;

FIG. 23 is a front view of an eye having multiple cavities formed using an ultra short pulse laser as shown in FIG. 21, the cavities forming a substantially ring-shaped configuration;

FIG. 24 is a front view of an eye having multiple cavities formed using an ultra short pulse laser as shown in FIG. 21, the cavities formed in an area offset from the main optical axis;

FIG. 25 is a side elevational view in cross section of the eye of FIG. 21 with a device applying a photosensitizer to the surface of the cornea;

FIG. 26 is a side elevational view in cross section of the eye of FIG. 25 with a reshaping device proximate to the external surface of the cornea;

FIG. 27 is a side elevational view in cross section of the eye of FIG. 26 with the reshaping device immediately adjacent the external corneal surface and a laser heating the cornea;

FIG. 28 is a side elevational view in cross section of the eye of FIG. 27 showing the cornea reshaped to conform to the predetermined shape of the reshaping device;

FIG. 29 is a side elevational view in cross section of the eye of FIG. 28 after the reshaping device has been removed; and

FIG. 30 is a side elevational view in cross section of a device according to another embodiment of the present invention.

### DETAILED DESCRIPTION OF THE PREFERRED EMBODIMENT

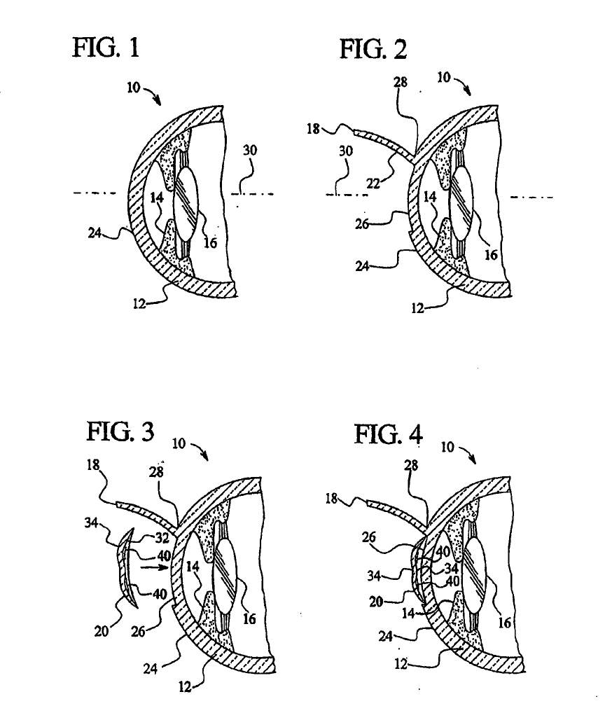

FIG. 1 is a side elevational view in cross section taken through the center of an eye 10, which includes a cornea 12, a pupil 14 and a lens 16. If the cornea 12 and lens 16 do not cooperatively focus light correctly on the retina (not shown) of the eye to thus provide adequate vision, the curvature of the cornea can be modified to correct the refractive power of the cornea and thus correct the manner in which the light is focused with respect to the retina.

As seen in FIGS. 1-7, the refractive properties of the eye can be modified or altered by forming a flap 18 in the surface 12 of the cornea, preferably by placing a reshaping device 20 having a predetermined shape on the surface 12 of the cornea, heating the reshaping device and in turn heating the surface of the cornea. However, it is noted that the cornea can be heated by any means suitable, such as directly by a laser or chemically or any other method that would allow heating the cornea to the proper temperature. Heating the cornea to the predetermined temperature causes the corneal stroma to soften and have a gel-like or gelatinous consistency. The gelatinous corneal portion then can flow and reform to take the form of the interior surface 32 of the reshaping device, thus changing the refractive properties of the cornea and the eye.

To begin, the refractive error in the eye is measured using wavefront technology, as is known to one of ordinary skill in the art. A more complete description of wavefront technology is disclosed in U.S. Pat. No. 6,086,204 to Magnate, the entire content of which is incorporated herein by reference. The refractive error measurements are used to determine the appropriate shape of lens or contact 20 to best correct the error in the patient's cornea. Preferably, the lens 20 is manufactured or shaped prior to the use of the wavefront technology and is stored in a sterilized manner until that specific lens shape or size is needed. However, the information received during the measurements from the wavefront technology can be used to form the lens using a cryolathe, or any other desired system or machine.

Preferably, a flap or portion 18 can be formed in the surface 24 of the cornea 12, as seen in FIG. 2. Preferably, the flap is formed in the stromal layer of the cornea, but does not necessarily need to be formed in the stromal layer and can be formed in any desired portion of the cornea. The flap may be formed be any means desired, such as with a knife, microkeratome, or with a laser. Preferably, an internal area of the cornea is separated into first and second substantially circular shaped internal surfaces 22 and 26, respectively, to form the circular shaped corneal flap 18. First internal surface 22 faces in a posterior direction of cornea 12 and the second internal surface 26 faces in anterior direction of the cornea 12. The flap 18 preferably has a uniform thickness of about 10-250 microns, and more preferably about 80-100 microns, but can be any suitable thickness. A portion 28 of flap 18 preferably remains attached to the cornea by an area at the periphery of the flap. However, the flap can be any suitable configuration, such as a flap attached to the cornea at a location other than at the periphery or a flap that is not attached to the cornea at all. Additionally, the flap may be shaped or sized as desired and does not need to be circular.

The flap is moved or pivoted about portion 28 using any device known in the art, such as a spatula or microforceps or any other device, to expose the first and second corneal surfaces 22 and 26, respectively. The flap preferably exposes a portion of the corneal surface that intersects the main optical axis 30 and allows uninhibited access thereto.

Lens or shield 20 can then be positioned adjacent and overlying the surface 22 of the cornea, as seen in FIG. 4. However, it is noted that the lens does not necessarily need to be positioned adjacent a surface exposed by a flap and may be positioned on the external surface 24 of the cornea 12 or the second internal surface 26. The surface exposed by the flap is the preferred method, since the cornea will not develop tissue necrosis, which may be possible, if the lens is positioned adjacent the external surface of the cornea.

Lens 20 is preferably any metal that can absorb heat and transmit and distribute heat throughout the lens in a uniform or substantially uniform manner. However, the lens does not necessarily need to be metal and can be any synthetic or semi-synthetic material, such as plastic or any polymer or any material that has pigmentation that would allow the lens to absorb the heat from the laser and transmit and distribute the heat uniformly throughout the lens.

Additionally, lens 20 is substantially circular and has a first or inner side or surface 32 and a second or outer side or surface 34 and preferably has a substantially concave shape. The lens preferably has a predetermined shaped, or more specifically, the first surface 32 preferably has a predetermined shape that would be the proper shape of the surface 26 of the cornea plus the flap 18 to focus light onto the retina. In other words, if the interior of the cornea were the shape of the interior surface of the lens the patient would be able to have 20/20 vision or better.

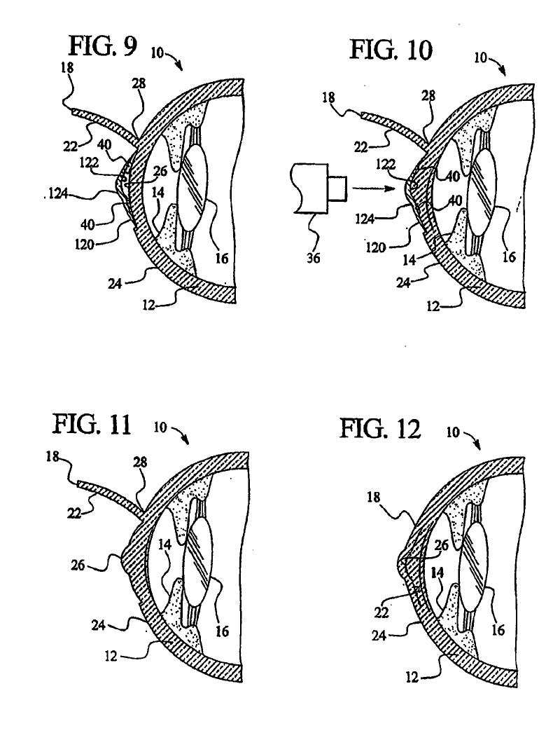

FIGS. 1-7 show the correction of myopic error using a concave lens 20. However, the lens can be formed such as lens 120, shown in FIGS. 8-12 and discussed below, for correction of hyperopic error or any other shape desired for the correction of astigmatic error or any other error.

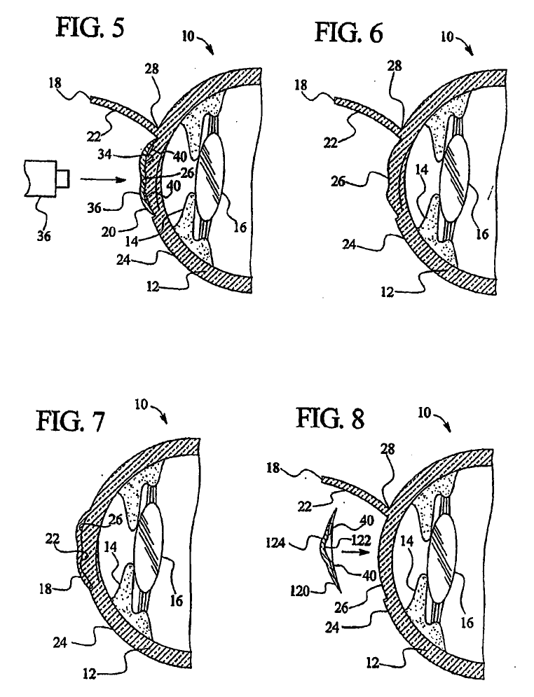

Once the reshaping device is positioned immediately adjacent the exposed surface 26 of the cornea 12, a heating device is applied or administered to the reshaping device 20, which in turn transfers the heat to the surface of the cornea. Preferably, as seen in FIG. 5, a laser 36 is aimed and fired or directed, so that the light emitted form the laser or the laser beam L is absorbed by the reshaping device 20 and then absorbed by or transferred to the cornea. Preferably, the laser beam is in the infrared portion of the electromagnetic spectrum, such as light supplied by a Nd-Yag laser at 1.32 μm, a Holmium laser at 2.2 μm or a Erb-Yag laser at 2.9 μm, or any other laser light wave length that is absorbed by water. For example, the laser light can be from a CO₂ laser or a visible light laser, such as an argon laser. Additionally, the reshaping device can be heated by any means suitable, such as microwaves.

The laser beam preferably heats the lens so that the inner surface of the reshaping device is about or below 60° Celsius (140° F.), which in turn heats the corneal surface 26 (preferably the stroma) to about the same temperature, thereby softening the cornea. The reshaping device inner surface temperature is constantly controlled or measured, preferably using multiple thermocouples 40 on the inner surface of the reshaping device. The thermocouples are linked to a computer control system (not shown) using any method known in the art, such as direct electrical connection or wires or a wireless system. The computer control system monitors the temperature and controls the laser to change the temperature of the reshaping device. The computer can maintain a precise constant temperature, increase temperature or decrease temperature as desired, and at any rate desired. This computer control system, along with the thermocouples ensure an adequate and precise temperature, since heating the cornea above 60° Celsius can cause coagulation of the cornea.

By heating the corneal stroma to about or below 60° C., the molecules of the cornea are loosened, and the cornea changes from a substantially solid substance to a gelatinous substance or gel-like substance. However, the corneal temperature is maintained at or below 60° C., and therefore, protein denaturization does not occur as with conventional thermal coagulation. Since the heated portion of the cornea is now flowable, the cornea reforms and is molded to take the shape of the inner surface 32 of the reshaping device, thereby forming the cornea into the reformed, corrected shape in an effort to provide the patient with 20/20 vision. The cornea is then cooled by applying cool or cold water, by applying air or by simply removing the heated reshaping device or the heat from the reshaping device and using the ambient air temperature. As the cornea cools, it is held by the reshaping device 20 to the preferred shape, which becomes its new permanent shape once the cornea is completely cooled and changes from its gel-like consistency to its original substantially solid consistency, as shown in FIG. 6.

The flap 18 is then replaced so that it covers or lies over the first surface 26 of the cornea 12 in a relaxed state, as seen in FIG. 7. This new permanent shape allows the cornea to properly focus light entering the eye on the retina. The refractive power of the eye is then measured to determine the extent of the correction. If necessary, the method can be repeated.

A reshaping lens can be applied to the external surface of the cornea, if necessary, after the flap has been replaced to maintain the proper corneal curvature or the eye can be left to heal with no additional reshaping lens being used.

Furthermore, at the end of the method, if desired, topical agents, such as an anti-inflammatory, antibiotics and/or an antiprolifrative agent, such as mitomycin or thiotepa, at very low concentrations can be used over the ablated area to prevent subsequent haze formation. The mitomycin concentration is preferably about 0.005-0.05% and more preferably about 0.02%. A short-term bandage contact lens may also be used to protect the cornea.

By reforming the cornea into the desired shape in this manner, a highly effective surgical method is formed that allows perfect or near perfect vision correction without the need to ablate any of the cornea or causing a gray to white response in the cornea of the eye.

### FIGS. 8-12

As shown in FIGS. 8-12, the same general method as shown in FIGS. 1-7 can be used to correct hyperopic error in the cornea. In this method, a substantially circular convex reshaping device 120, rather than concave reshaping device 20, having a first or inner surface 122 and a second or outer surface 124, is used and placed immediately adjacent and overlying the surface 26 of the cornea. A heating element, preferably a laser 36, is used to heat the reshaping device, which in turn increases the temperature of the cornea to about or below 60° Celsius, as described above. This heating causes the cornea to soften and turn into a gel-like material, thereby becoming flowable to conform to the inner surface 122. Once the corneal surface 26 is cooled and permanently reformed to the inner surface of the reshaping device, the device is removed and the flap replaced. The hyperopic error is corrected and the cornea can now effectively focus light on the retina, as described above.

This method for correcting hyperopic conditions is substantially similar to the method for correcting myopic conditions. Thus, the entire method described above for correcting myopic error of the cornea applies to the correction of hyperopic error, except for the exact configuration of the reshaping device.

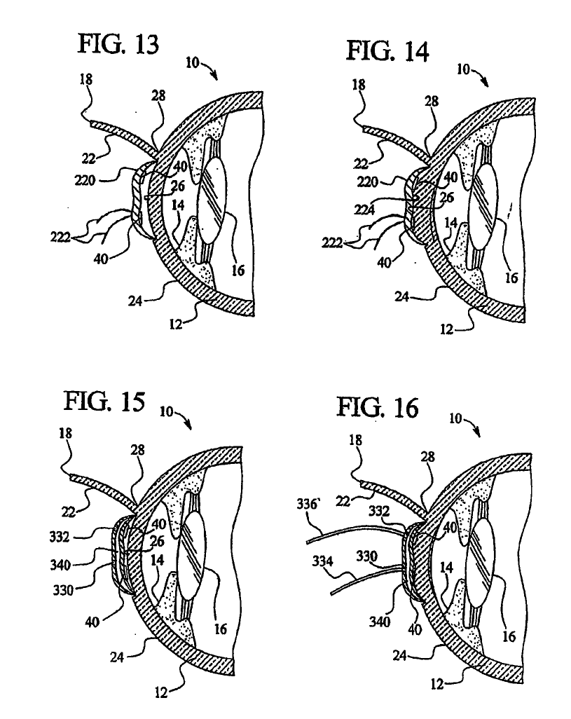

### FIGS. 13 and 14

As shown in FIGS. 13 and 14, the reshaping device can be a thermally conductive plate or reshaping device 220 that is electrically connected to a power source (not shown) using electrical wires 222. The thermally conductive plate 220 is preferably any metal or conductive material that can conduct electricity supplied by a power source (not shown) and turn the electricity into heat. Furthermore, the plate preferably is formed from a material that would allow an equal or substantially uniform distribution of heat through the plate.

This method is similar to those described above; however, the temperature of the cornea is increased using the thermocouple plate instead of a laser. As seen in FIG. 13, the plate 220 is heated to the desired temperature, preferably about or below 60° Celsius, as described above. This causes loosening of the corneal molecules or softening of the cornea, which allows the cornea to conform to surface 224 of plate 220, thereby permanently changing the shape of the cornea. Once the corneal surface 26 has cooled and permanently reformed to the inner surface of the thermocouple plate, the plate is removed and the flap replaced. The cornea can now effectively focus light on the retina, as described above.

Although, the method is shown in FIGS. 13 and 14 using a thermally conductive plate to correct myopic error, a thermally conductive plate can be used to change the shape of the cornea in any manner desired, such to correct astigmatic or hyperopic error in the cornea.

Furthermore, since this method is substantially similar to the methods described above, the description of those methods and references numerals used therein, excluding the specific lens and heating element, apply to this method.

### FIGS. 15 and 16

As shown in FIGS. 15 and 16, reshaping device 320 can be a container, i.e., hollow, with an irrigation port 330 and an aspiration port 332 providing access to interior chamber 340. Reshaping device 320 is preferably any metal or plastic that can be filled with a liquid and absorb heat and distribute the heat throughout the reshaping device in a uniform or substantially uniform manner. However, the reshaping device does not necessarily need to be metal and can be any synthetic or semi-synthetic material, such as plastic or any polymer of any material that would allow the lens to absorb the heat from the liquid and distribute the heat uniformly throughout the reshaping device.

The method of FIGS. 15-16 is similar to those described above; however, the temperature of the cornea is increased using a tube 334 that couples to the irrigation port and fills chamber 340 of the container with a liquid of a predetermined temperature, preferably about or below 60° Celsius (140° F.). Once filled with the liquid, the inner surface of the reshaping device would increase to the desired temperature, thereby loosening the molecules of the cornea or softening surface 26 of the cornea, which allows the cornea to conform to surface 324 of reshaping device 320 and results in the proper reformation of the cornea. The liquid can then be removed from the container via the aspiration tube 236, allowing the cornea to cool and permanently reform to the desired shape, as described above. Once the corneal surface 26 has cooled and permanently reformed to the inner surface of the reshaping device, the reshaping device is removed and the flap replaced. The cornea can now effectively focus light on the retina, as described above.

Although, the method shown in FIGS. 15 and 16 uses a container to correct myopic error, this method can be used to change the shape of the cornea in any manner desired, such to correct astigmatic or hyperopic error in the cornea.

Furthermore, since this method is substantially similar to the methods described above, the description of those methods along with the reference numerals used therein, excluding the specific reshaping device and heating element, apply to this method.

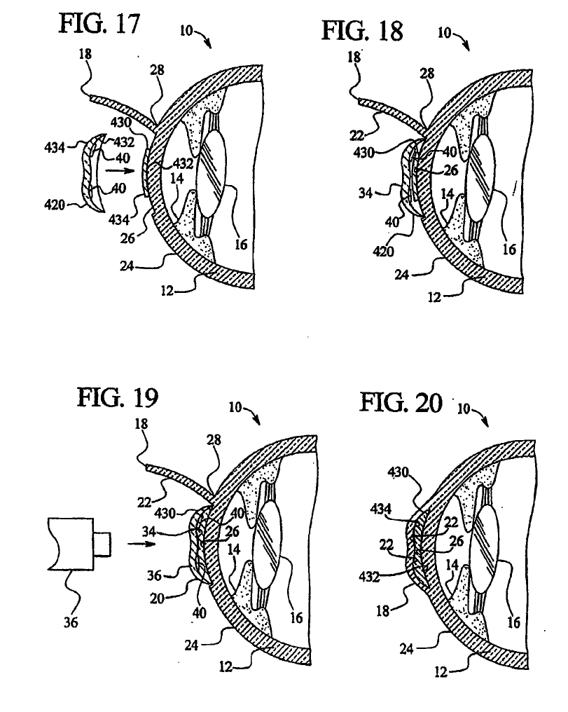

### FIGS. 17-20

As seen in FIGS. 17-20, a modified method does not necessarily need to be performed on the cornea, but can be performed on a separate lens or inlay 430. Inlay 430 is preferably a substantially circular polymeric or synthetic inlay or blank that has a predetermined thickness and a first side 432 and a second side 434 and is positioned under the flap adjacent second surface 26 to correct refractive error in the eye. For a more complete description of use of an inlay, see U.S. Pat. No. 6,197,019 to Peyman, the entire contents of which are herein incorporated by reference.

As described above and seen in FIGS. 18 and 19, a reshaping device 420 having a first surface 422 and a second surface 424 is placed over the inlay 430 adjacent first second surface 434 and heated to the appropriate temperature using a laser 36. Since the inlay is a polymer and is not formed from living cells, there is no need to keep the temperature at or about 60° Celsius (140° F.). The rise in temperature of the lens causes the inlay 430 to soften or become a gelatinous material and thereby flowable which allows the inlay to conform to the shape of the inner surface 422 of reshaping device 420. In a similar manner to that described for the cornea above.

As seen in FIG. 20, once the reshaping device 420 is removed, the flap 18 is placed over the inlay 430. First internal surface 22 is positioned so that it overlies the second surface 434 of inlay 430 without substantial tension thereon. In other words, the flap is merely laid overtop of the inlay 430 so as to not cause undue stress or tension in the flap and possibly causing damage thereto.

It is noted that the method of FIGS. 17-20 is not limited to the first herein described method using a reshaping device and a laser, but can be used with any heating means, such as the container method and the thermally conductive plate method also described herein and any other method that would heat a reshaping device overlying the inlay to the appropriate temperature.

Additionally, this method of FIGS. 17-20 can be preformed with a lens that has a predetermined refractive index, is a blank having no refractive index or a lens that has been modified by a laser, a cryolathe or any other method known in the art to have a predetermined refractive index. For example, with a blank, the inlay can have no refractive power, the entire corrective change in the lens coming from the conformation to the inner surface of reshaping device 420 or the inlay can have refractive power with the reshaping device 420 simply modifying the refractive properties.

Although, the method shown in FIGS. 17-20 uses a lens to correct myopic error, this method can be used to change the shape of the cornea in any manner desired, such to correct astigmatic or hyperopic error in the cornea.

Furthermore, since this method is substantially similar to the methods described above, the description of those methods along with the reference numerals used therein applies to this method.

### FIGS. 21-29

FIGS. 21-29 illustrate another embodiment of the present invention for correcting refractive error in the eye, wherein a laser 500, such as a short pulse laser, is used to form cavities or three dimensional portions 502 in the cornea 12 of an eye 10. A mold or lens 504 is then used to reshape the cornea to correct the refractive error in the eye.

First, as described above the refractive error in the eye is measured using wavefront technology, as is known to one of ordinary skill in the art or any other suitable method. The refractive error measurements are used to determine the appropriate shape of lens or contact 504 to best correct the error in the patient's cornea 12. Preferably, the lens or reshaping device 504 is manufactured or shaped prior to the use of the wavefront technology and is stored in a sterilized manner until that specific lens shape or size is needed. However, the information received during the measurements from the wavefront technology can be used to form the lens using a cryolathe, laser, or any other desired system, method or machine.

Preferably lens 504 is preferably clear and formed any organic, synthetic or semi-synthetic material or combination thereof, such as plastic or any polymer or any material that has pigmentation that would allow laser light to pass therethough such that laser light could heat the cornea as described herein. Lens 504 has a first surface 520 and a second surface 522. The second surface preferably is adapted to be positioned adjacent a surface of the cornea and has a predetermined curvature that will change the curvature of the cornea to correct refractive error. However, the lens does not necessarily need to be formed in this manner and can be opaque and/or formed in any manner described above or in any manner suitable for changing the curvature of the cornea.

As shown in FIG. 21, the laser 500 is preferably fired at a portion 506 of the cornea beneath or under the exterior surface 24 of the cornea, forming a predetermined pattern of cavities, which have a predetermined size and shape. In other words, the laser 500 is preferably fired at the stromal layer of the cornea. The laser is programmed to form up to 10,000 small cavities or three dimensional aberrations 502 in the stroma of the eye. Each cavity has a diameter of about 10 microns or less to about 1 millimeter. It is noted that cavities 502 do not necessarily need to be formed in the stroma and can be formed in any portion of the cornea, such as in the Bowman's layer, the epithelial layer, or suitable portion of the eye or any combination thereof.

Laser 500 is preferably an ultra short pulse laser, such as a femto, pico, or attosecond laser; but may be any light emitting device suitable for creating cavities 502. The ultrashort pulse laser 500 is positioned in front of the eye and focuses the laser beam in the cornea 12 at the desired depth for creating multiple cavities. Ultra short pulse lasers are desired since they are capable of ablating or vaporizing corneal tissue beneath the surface of the cornea without disrupting, damaging or affecting the surface of the cornea. Additionally, ultra short pulse lasers are high precision lasers that require less energy than conventional lasers to cut tissue and do not create “shock waves” that can damage surrounding structures. Cuts or ablation performed using ultra short pulse lasers can have very high surface quality with accuracy better than 10 microns, resulting in more precise cuts than those made with mechanical devices or other lasers. This type of accuracy results in less risks and complications than the procedures using other lasers or mechanical devices. However, it is noted that the cavities 502 can be formed by any manner or device desired.

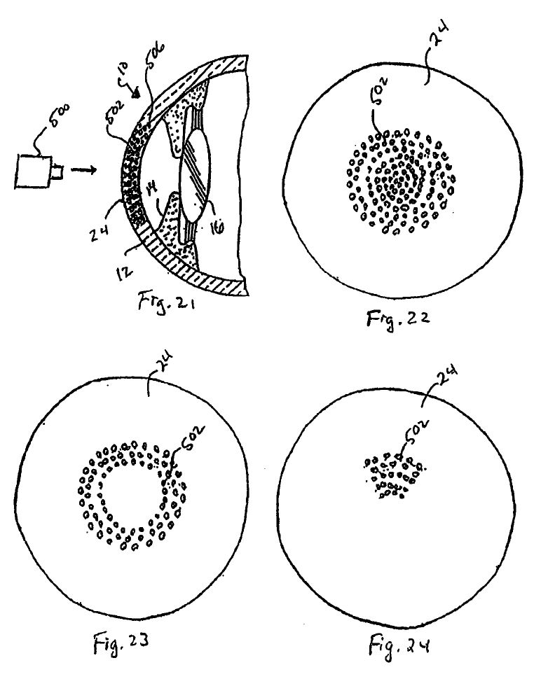

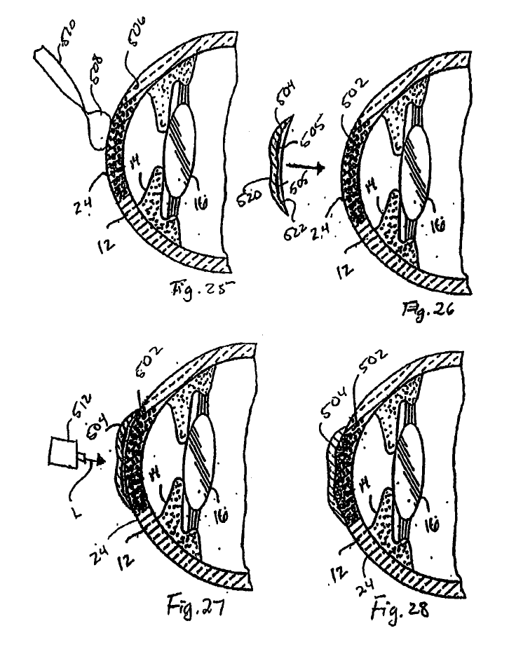

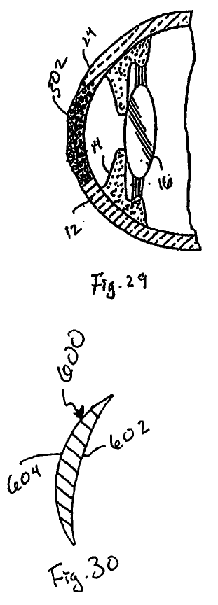

As shown in FIGS. 22-24, cavities 502 can form various configurations or patterns. For example, the cavities can form a substantially circular pattern (FIG. 22), a substantially ring-shaped pattern (FIG. 23), or a pattern that is offset from the main optical axis (FIG. 24). Each specific configuration is particularly useful for correcting a specific vision problem in the eye. For example, a substantially circular pattern facilitates correction of myopia and hyperopia, a substantially ringed shaped pattern facilitates correction of presbyopia and a pattern offset from the main optical axis facilitates correction of astigmatism. It is noted that these patterns and configurations are exemplary purposes only and the cavities can be formed in any suitable configuration for correcting myopia, hyperopia and/or astigmatism or any other refractive error in the eye.

As shown in FIG. 25 a photosensitizer or an ultraviolet absorbing compound 508 can be applied to the surface of the cornea 24 using a device or applicator 510. The plhotosensitizer can be applied to the entire cornea or merely to specific areas and can absorb ultraviolet or near ultraviolet red radiation to help facilitate or create cross-linking of collagen and hold the corneal structure into the new reformed shape. A suitable material for photosensitizing the cornea is riboflavin. Additionally, photosensitizer 508 is preferably a liquid or gel that is capable of initiating or catalyzing the energy from the laser 500; however, the photosensitizer can be any suitable substance. Furthermore, the initiator does not necessarily need to be a photosensitizer and can be any suitable substance that facilitates formation of the cavities or reduces the heat and/or energy required to form the cavities 502.

Once the photosensitizer is applied and allowed to spread through or penetrate to the corneal stroma, lens or reshaping device 504 is positioned immediately adjacent the external corneal surface, as shown in FIGS. 26 and 27. Reshaping device second surface 522 which has a predetermined curvature is preferably positioned immediately adjacent the external surface of the cornea, overlying all or substantially all of the cavities 502; however, it is noted that it is not necessary for the reshaping device to overlie all or substantially all of the cavities 502 and can overlie only a portion of the cavities 502 if desired. The reshaping device 504 is substantially similar to the embodiments described above and any description thereof is application to the present embodiment, including the use of thermocouples 505.

As shown in FIG. 28, laser or light emitting device 512 is aimed and fired at the corneal stroma, at or approximately at the portion of the cornea in which the cavities 502 are formed. Laser 512 can be the same laser, or a substantially similar laser, as laser 500, it can be any device capable of emitting ultraviolet light or near ultraviolet red radiation or laser 512 can be any suitable laser or light emitter. The laser beam L (preferably combined with the reaction from photosensitizer 508) then heats the corneal stroma to above body temperature and below a temperature at which coagulation occurs, preferably at about 60° C., and preferably to between about 45° C.-50° C. The preferred temperatures allow or facilitate cross-linking of the collagen cells in the eye, so that the cornea can be reshaped more easily. As with the embodiments described above, the temperature can be controlled using the thermocouples and a suitable computer control system.

Additionally, it is noted that the laser can heat the reshaping device, which in turn heats the cornea, or the cornea can be heated in any manner described herein.

By heating the corneal stroma to about or below 60° C., the molecules of the cornea are loosened, and the cornea is softened, in a manner substantially similar to that described above. However, the corneal temperature is maintained at or below 60° C., and therefore, protein denaturization does not occur as with conventional thermal coagulation. Since the heated portion of the cornea is now softened, the cornea reforms and is molded to take the shape of the inner surface of reshaping device 504, thereby forming the cornea into the reformed, corrected shape in an effort to provide the patient with 20/20 vision. The cornea is then cooled by applying cool or cold water, by applying air, by letting the reshaping device 504 cool through time or by simply removing the heated reshaping device or the heat from the reshaping device and using the ambient air temperature.

Preferably, as the cornea cools, it is held by the reshaping device 504 to the preferred shape, which becomes its new permanent shape once the cornea is completely cooled and changes to its original substantially solid consistency, as shown in FIG. 29.

Preferably, the reshaping device 504 is transparent as described above, thus allowing the patient to see while the reshaping device is still on the external surface of the eye. In other words, as the cornea cools, the reshaping device 504 acts as a contact lens.

It is noted that reshaping device does not necessarily need to be applied to the external surface of the cornea and can the positioned directly on the Bowman's layer, directly on the corneal stroma or any other suitable portion of the cornea. This positioning can be achieved by forming a flap that would expose the desired portion of the internal structure of the cornea. As described herein the flap can be a Lasik type flap (i.e., attached to the cornea at the periphery—see. FIG. 3), or it can be a flap that is attached at a central portion of the cornea (i.e., along the main optical axis), the flap can be completely removed, or the internal structure of the cornea can be exposed in any other suitable manner.

In another embodiment, device 600 (FIG. 30) can be used in Glaucoma therapy. Heat can be applied to the exterior of the cornea adjacent or in proximity to the Schlem's canal and trabecular meshwork. Preferably, the device is positioned on the external portion of the cornea adjacent the Schlem's canal and/or the trabecular meshwork, but can be positioned externally or internally in any suitable manner. This device generally requires no invasive procedures to be performed on the eye, thus reducing the risk of possible damage.

Such application of the device 600 encourages absorption of deposits which plug the out flow of the aqueous fluid. The heat damages (or kills) certain cells and encourages regeneration of the cells (i.e., re-population), while simultaneously causing other cells to become more active, thereby facilitating removal of debris. The device preferably heats the corneal stroma to above body temperature and below about 60° C., and preferably to between about 45° C.-50° C.; however, the stroma can be heated to any suitable temperature. As with the embodiments described above, the temperature can be controlled using thermocouples and/or a suitable computer control system or in any other suitable manner.

The device 600 can be substantially circular, substantially semicircular, substantially ring shaped, arcuate or any other suitable configuration that would facilitate or achieve the desired outcome. Device 600 can be between about 10 mm to about 14 mm in diameter; but can be any suitable size. Preferably, the device has a first surface 602 and a second surface 604. The first surface is generally arcuate and has a radius of curvature of about the same curvature as the external surface of the eye; however, the device can have any suitable configuration. The first surface is preferably positioned on the external surface of the cornea at or near the desired area. The device can be heated using any desired means, such as electrically, with lasers and/or water or any suitable means or any combination of the herein described means or any other suitable means.

Once the device is heated, the eye can be monitored for any suitable duration to determine if any blockage in the Schlem's canal and/or the trabecular meshwork has been reduced or relieved. If desired, the procedure can be repeated one or multiple times or until the desired result is achieved.

Furthermore, a laser can be used to heat the appropriate portion of the eye. For example, a laser can be used to ablate or heat the meshwork, thus enhancing the outflow of vitreous fluid. The laser can be used alone to heat/ablate portions of the eye (e.g., the meshwork) or in combination with any other device or method described herein. The addition of the laser to the system allows the light to penetrate deeper into the cornea (or other portion of the eye) while controlling the temperature at the application site. The laser can be applied simultaneously from outside (through the conjunctiva and or sclera) or in a non-coagulative form to the Schlem's canal (at the junction of the cornea and sclera) in treatment of glaucoma.

The device 600 can also be used to treat small choroidal tumors, melanoma and/or Retinoblastoma, among other things. In this embodiment, the device is positioned adjacent (or in any suitable location) the choroidal tumors, melanoma and/or Retinoblastoma and heat is applied. The heat is preferably controlled to be below 60 degree Celsius to prevent coagulation of the tissue while achieving destruction of the tumor cells which are more sensitive to heat application than normal cells; however, the heat can be within any suitable range including any temperature above body temperature and at or below the temperature at which coagulation occurs. Preferably the temperature to which the device (and thus the temperature of the specific area of the eye) is heated is closely controlled or monitored using computers and/or any other means, as described above, or by any suitable means or device.

The heat to treat small choroidal tumors, melanoma and/or Retinoblastoma, among other things, is applied alone through device 600 or in conjunction with a laser. The laser can be any suitable laser, including a laser within the visible spectrum or any other suitable wavelength. Furthermore, the device can be used with an ultrasonic device or radio frequency probe or any other suitable device.

Additionally, the device 600 can have any suitable diameter to eliminate or treat any size tumor. For example, the device can be substantially circular with a diameter of about 2-3 mm to treat smaller tumors or the device can be 3-7 mm to eliminate or treat larger tumors. However, it is noted that the device can be any configuration and/or size disclosed above or any other suitable size and/or configuration.

Furthermore, the device 600 can be used alone or in a combined system for treatment of Age Related Macular Degeneration (ARMD). As with several of the embodiments described above, it is preferable not to increase the corneal tissue temperature above 59 degrees Celsius (or more preferably to above 50 degrees Celsius); however, the cornea can be heated to any suitable temperature between about body temperature and about 60 degrees Celsius. Preferably the temperature that the macula is heated to is closely controlled or monitored using computers and/or any other means, as described above or by any suitable device or means.

Additionally, device 600 can be used simultaneously or substantially simultaneously with a device that also applies heat through electricity, ultrasound, radio frequency wave or a laser in visible or infrared light or heated water. The application time of the heat is generally between about 5 seconds to about 600 seconds, but can be 1000 seconds or more. The spot size is preferably between about 0.1 to about 10 mm or larger, but can be any suitable spot size. The diameter of device 6—when treating ARMD is preferably between about 10 to about 15 mm and is preferably substantially ring shaped; however, the instrument can have any suitable shape or configuration and be any suitable size.

Furthermore, if desired the above method can be used to heat the macular simply using a device that applies heat through electricity, ultrasound, radio frequency wave or a laser in visible or infrared light or heated water without device 600 to treat ARND.

Additionally, any of the above described methods and/or devices can be used to encouraging the penetration of a drug applied to an adjacent tissue. For example, the drug (or any other substance) can be applied topically to the cornea of the eye or in any suitable or desired area of the body. Examples of substances that can be applied are photosensetizers, antimetabolites, anti-cancer, ant-inflammatory, antibiotics, macrolides, antiprostoglandins etc. It is noted that the present method is not limited to these substances and any suitable substance can be used to treat the appropriate portion of the body or to benefit the human body or facilitate healing thereof.

In another embodiment of the present invention, a system is configured to use a device to apply controlled heat substantially simultaneously or simultaneously with a drug or medicinal substance on or in a desired portion of a patient or animal. For example, a medicinal substance and controlled heat can be applied on the skin, on or in the nose, on or in the mouth, on the mucosa, on or in any tissue, outside or inside the eyelid, on the cornea, conjunctiva or sclera or any other suitable portion of the patient.

Preferably, the system includes a device that is configured to be positioned adjacent a desired external portion of a patient, a heating element or means, a computer control system and a medicinal substance delivery system; however, the system does not need to include each of or only these elements.

The device can be positioned adjacent or immediately covering an appropriate portion of the body or be positioned in any other suitable manner. The device is preferably formed from a suitable material that would allow efficient heat absorption and transfer. For example, the device can be made from metal or from any suitable synthetic or semi-synthetic material, such as plastic or any polymer or any material that would allow the device to absorb the heat and transmit and distribute the heat uniformly throughout the device or from any suitable materials described above. The device can be applied in or on any suitable location. For example, the device can be applied on the eyelid, on the sclera, on the skin or any other place on a patients' body. The device can be any device described herein, have any suitable configuration, or can have any suitable heating device or element. Preferably, the device is sized and configured for a specific local application. For example, the devices described above can have concaved surfaces for placement on the external surface of the cornea.

The heating element or means can be any suitable device for heating the device. The heating means can be coupled to the device, implanted in the device, be an integral part of the device or be located in a position relative to the device. For example, the heating means can be an electrical circuit or current supplied to a circuit or system on or in the device, a system or component imbedded or integral with the device or the heating means can be an external system configured to transfer heat to the device. The heating means is preferably in electrical connection and/or communication with the computer control system, but can be activated in any suitable manner. However, as stated above, the heating means can be any suitable device and can heat the device in any suitable manner. For example, the heating means can use radiation, light, ultrasound, or electrical current, alone or in any suitable combination.

Preferably, the heat application is controlled in any of the suitable manners described above, which facilitates penetration of medication or medicinal substance (or other substance) in the eye (or other suitable location), including the surrounding tissue.

The computer control system is preferably electrically coupled to the device and the heating means and is used to monitor and control the temperature of the device. The computer control system preferably is capable of precisely calculating or monitoring the temperature of the portion of the body and controls the temperature thereof to achieve favorable results. However, it is noted that the computer control system can be any suitable system and does not necessarily need to be coupled to each or either of these devices and can be coupled to one or none and monitor the system wirelessly or in any other manner.

The combination of heat and medicinal substance application will enhance penetration of the medicinal substance inside the tissue. The heat can be precisely controlled using any type of temperature sensors (e.g., theremocouples) and the computer control system. Preferably the temperature is controlled within a range plus or minus about ten degrees Celsius of body temperature. For example, the temperature can be controlled to about 25 degrees Celsius to about 45 degrees Celsius or any suitable range or temperature therein. More preferably, the temperature can be controlled to about 35 degrees Celsius to about 41 degrees Celsius or any suitable range or temperature therein; however, the temperature can be heated to any suitable specific temperature or range of temperatures.

Preferably the medicinal substance is applied topically; however, it can be administered in any suitable manner. The amount and rate at which the substance is administered may be computer controlled or done manually. The medicinal substance can be any suitable compound. For example, the drugs or medicinal substances can be antibiotics, anti-fungals, antivirals, anti-cancers, anti-inflammatories, anti-prostaglandins, steroids, hormones, growth factors NGF, GGF, any desired enzyme, Anti-VEGF, Anti-PDGF, VEGF, Anti-glaucoma and siRNA, nano particles, Antibodies, macrolides, and/or all hydrophilic or lipophilic compounds.

The device can also be implanted into the body and externally heated using a suitable device. For example, the device can be heated using a remote control system or other device capable of penetrating the appropriate portion of the body. The device can be configured with an internal heating element (or any type of heating means described above) that is activated externally (e.g., via an external wireless signal) or the device can absorb heat that is transferred thereto via an external source (e.g., via a laser, radiation or any other suitable source).

The device can be surgically implanted or injected or positioned within the patient in any other suitable manner. For example, the device can be positioned in the eye as a corneal inlay (e.i., inserted between layers of cornea (e.g., under a corneal flap or in a corneal pocket)), an intraocular lens, or positioned in any other manner in the eye or in any other suitable portion of a patients' body. When inserted between layers of the cornea (or otherwise in a patient's body), the inlay can be heated by directed contact with a heating element (e.g., probes or wires inserted through the cornea) or remotely (e.g., a laser). If desired, a flap can be formed (or reformed) to allow access to the device for heating purposes.

As with the externally applied device described above, the implanted device is preferably formed from a suitable material that would allow efficient heat absorption and transfer. For example, the device can be made from metal or from any suitable synthetic or semi-synthetic material, such as plastic or any polymer or any material that would allow the device to absorb the heat from the laser and transmit and distribute the heat uniformly throughout the device or from any suitable materials described above. Furthermore, when inserted in the eye, the inlay or device is formed from suitable transparent materials and can have refractive powers or the ability to alter the refractive power or the eye, as described above.

Additionally, if desired the implanted device can be combined with slow release drug delivery system. Such a combined system would be useful for delivery of pain medication or other medications to desired area inside the body, among other uses. Suitable drugs could be administered through drops, ointment, slow release, microspheres, nano-particles, compacted nano-particles, viral factor, and any other suitable or desired drugs.

Preferably, as described above, a computer control system monitors and controls the temperature of the overall system to precisely control the temperature at the site within the body, and the description of the externally positioned device is applicable to the implanted device. Furthermore, if desired, the computer control system can control the release of the drug.

Any of the herein described embodiments can be used with any combination of the other embodiments. For example, applying heat to encourage drug penetration and/or relieving internal pressure in the eye can occur simultaneously or substantially simultaneously as the embodiments describing reshaping of the cornea.

While various advantageous embodiments have been chosen to illustrate the invention, it will be understood by those skilled in the art that various changes and modifications can be made therein without departing from the scope of the invention as defined in the appended claims.

What is claimed is:

1. A system for treating a patient, comprising:
a device configured to be positioned adjacent a portion of the patient;
a medicinal substance delivery device, adapted to delivery a medicinal substance to the portion of the patient;
a heating means adapted to provide heat to said device; and
a control system configured to monitor said heating means and control the temperature of said device at the portion of the patient.

2. A system according to claim 1, wherein
said control system is a computer control system.

3. A system according to claim 1, wherein
said device is configured to be positioned on a portion of a patient selected from the group consisting of;
the cornea, the sclera and the skin.

4. A system according to claim 1, wherein
said device is configured to be positioned between layers of the cornea.

5. A system according to claim 4, wherein
said device is substantially circular.

6. A system according to claim 1, wherein
said control system is configured to control the temperature of the device, such that the portion of the patient is heated to between about 25 degrees Celsius and about 35 degrees Celsius.

7. A system according to claim 1, wherein
said device is configured to alter the refractive properties of the eye.

8. A system according to claim 1, wherein
said medicinal substance is a substance selected from the group consisting of antibiotics, anti-fungals, antivirals, anti-cancers, anti-inflammatories, anti-prostaglandins, steroids, hormones, growth factors, NGF, GGF, enzymes, Anti-VEGF, Anti-PDGF, VEGF, Anti-glaucoma, siRNA, nano particles, Antibodies, macrolides, hydrophilic compounds and lipophilic compounds.

9. A system according to claim 1, wherein
said heating means is selected from the group consisting of radiation, light, ultrasound, or electrical current.

10. A method of treating a patient, comprising the steps of
positioning a device adjacent a portion of a patient,
delivering a medicinal substance to said portion of the patient,
heating the device,
monitoring the temperature at said portion of the patient, and
controlling the temperature of the device.

11. A method according to claim 10, wherein
said step of controlling the temperature includes controlling the temperature using a computer control system.

12. A method according to claim 10, wherein
said heating step includes heating a device configured to be positioned on the surface of the cornea.

13. A method according to claim 12, wherein
said positioning step includes positioning a device that is substantially circular.

14. A method according to claim 10, wherein
said controlling step includes controlling the temperature of the device, such that the portion of the patient is heated to between about 25 degrees Celsius and about 35 degrees Celsius.

15. A method according to claim 10, wherein
said delivering step includes delivering a medicinal substance selected from the group consisting of antibiotics, anti-fungals, antivirals, anti-cancers, anti-inflammatories, anti-prostaglandins, steroids, hormones, growth factors, NGF, GGF, enzymes, Anti-VEGF, Anti-PDGF, VEGF, Anti-glaucoma, siRNA, nano particles, Antibodies, macrolides, hydrophilic compounds and lipophilic compounds.

16. A method according to claim 10, wherein
said heating step includes heating the device using at least one of the following: radiation, light, ultrasound, or electrical current.

17. A system for treating a patient, comprising:
a device configured to be implanted in a portion of an animal;
a heating means for heating the device to an appropriate temperature; and
a control system configured to monitor said heating means and control the temperature of said device at the portion of the patient.

18. A system according to claim 17, wherein
said heating means to coupled to said device and implanted in the portion of the animal; and
said control system includes an external remote device configured to send a signal to said heating means.

19. A system according to claim 17, wherein
said heating means is external to said animal; and
said control system is adapted to control said heating means such that said heating means transfers heat to said device.

20. A system according to claim 19, wherein
said heating means is a device selected from the group consisting laser or radiation.

21. A system according to claim 17, further comprising
a drug delivery system.

22. A system according to claim 21, wherein
said drug delivery system is selected from the group consisting of drops, ointment, microspheres, nano-particles, compacted nano-particles and viral factor.

23. A method for treating a patient, comprising the steps of
implanting a device in a portion of an animal,
heating the device to an appropriate temperature,
monitoring the temperature of the device, and
controlling the temperature of said device at the portion of the patient.

24. A method according to claim 23, further comprising the step of
administering a drug at said portion of the animal.
